(19) United States
(12) Patent Application Publication
(10) Pub. No.: US 2025/0243534 A1
(43) Pub. Date: Jul. 31, 2025

(54) SYSTEM AND METHOD FOR GENOTYPING STRUCTURAL VARIANTS

(71) Applicant: Life Technologies Corporation, Carlsbad, CA (US)

(72) Inventors: Krishna Reddy Gujjula, Georgetown, TX (US); Cheng-Zong Bai, Fremont, CA (US); Haktan Suren, Leander, TX (US)

(21) Appl. No.: 19/087,979

(22) Filed: Mar. 24, 2025

Related U.S. Application Data
(63) Continuation of application No. PCT/US2023/075334, filed on Sep. 28, 2023.
(60) Provisional application No. 63/412,001, filed on Sep. 30, 2022.

(51) Int. Cl. C12Q 1/6858

(52) U.S. Cl. CPC C12Q 1/6858; C12Q 2600/156

(57) ABSTRACT

Methods for determining genotypes of structural variants in a sample genome, may include: amplifying nucleic acid sequences at targeted locations in the sample genome by a panel targeting a plurality of structural variant marker to generate sequence reads; mapping the sequence reads to a modified reference genome to produce aligned sequence reads, wherein the modified reference genome includes a wild-type target region and a structural variant target region; for each structural variant marker, determining a read count for a wild-type allele and a read count for a structural variant allele; determining a probability for each possible genotype, wherein the possible genotypes include a homozygous wild-type genotype, a heterozygous genotype and a homozygous structural variant genotype; and selecting the genotype with a maximum probability value to provide an estimated genotype corresponding to the structural variant marker of the sample.

### CROSS-REFERENCE TO RELATED APPLICATIONS

This application is a continuation of International Application no. PCT/US2023/075334, filed Sep. 28, 2023, which claims the benefit under 35 U.S.C. § 119(e) of U.S. Provisional Application No. 63/412,001, filed Sep. 30, 2022. The entire contents of the aforementioned applications are incorporated by reference herein.

### FIELD

The present application relates to methods, systems, and computer-readable media for genotyping structural variants (SV) markers in samples using targeted sequencing and next-generation sequencing (NGS) technology.

### BRIEF DESCRIPTION OF THE DRAWINGS

The novel features of the invention are set forth with particularity in the appended claims. A better understanding of the features and advantages will be obtained by reference to the following detailed description that sets forth illustrative embodiments and the accompanying drawings of which:

FIGS. 1A-1C give examples of possible forward primer and reverse primer positions in wild-type alleles and variant alleles for types of structural variants.

FIGS. 2A-2C give examples of alignments of sequence reads with the wild-type allele target region and the variant allele target region for various genotypes of a deletion structural variant.

FIG. 3 is a block diagram of an example process for determining genotypes of structural variants.

FIG. 4 is a block diagram of an example process for determining the genotype for each structural variant marker per sample.

FIG. 5 gives a table of results of tests for genotyping canine SV markers.

FIG. 6 is a schematic diagram of an exemplary system for reconstructing a nucleic acid sequence, in accordance with various embodiments.

FIG. 7 is an example of a block diagram of an analysis pipeline for signal data obtained from a nucleic acid sequencing instrument.

### DETAILED DESCRIPTION

In accordance with the teachings and principles embodied in this application, new methods, systems and non-transitory machine-readable storage medium are provided for genotyping structural variant markers with known breakpoints using NGS technology. The teachings further provide for genotyping structural variants on a per sample per marker basis.

Advantages of the methods described herein include the ability to detect any type of structural variant (e.g., insertions, deletions, inversions, etc.) where a unique amplicon can be designed for an alternate allele of the particular structural variant. Multiple amplicons can be used to detect an allele. The methods described herein can genotype structural variants using targeted sequencing technology and NGS technology.

In various embodiments, a “structural variant” (SV) refers to a variation in the structure of a chromosome. Structural variants can include deletions, duplications, copy-number variants, insertions, gene fusions, inversions and translocations.

As used herein, structural variants are variants of size larger than 50 base pairs (bp). The primer design for a structural variant may have two amplicons per marker, one for wild-type/reference allele and another for mutant/variant/alternate allele.

In various embodiments, DNA (deoxyribonucleic acid) may be referred to as a chain of nucleotides consisting of 4 types of nucleotides; A (adenine), T (thymine), C (cytosine), and G (guanine), and that RNA (ribonucleic acid) is comprised of 4 types of nucleotides; A, U (uracil), G, and C. Certain pairs of nucleotides specifically bind to one another in a complementary fashion (called complementary base pairing). That is, adenine (A) pairs with thymine (T) (in the case of RNA, however, adenine (A) pairs with uracil (U)), and cytosine (C) pairs with guanine (G). When a first nucleic acid strand binds to a second nucleic acid strand made up of nucleotides that are complementary to those in the first strand, the two strands bind to form a double strand. In various embodiments, “nucleic acid sequencing data,” “nucleic acid sequencing information,” “nucleic acid sequence,” “genomic sequence,” “genetic sequence,” or “fragment sequence,” or “nucleic acid sequencing read” denotes any information or data that is indicative of the order of the nucleotide bases (e.g., adenine, guanine, cytosine, and thymine/uracil) in a molecule (e.g., whole genome, whole transcriptome, exome, oligonucleotide, polynucleotide, fragment, etc.) of DNA or RNA.

In various embodiments, a “polynucleotide”, “nucleic acid”, or “oligonucleotide” refers to a linear polymer of nucleosides (including deoxyribonucleosides, ribonucleosides, or analogs thereof) joined by internucleosidic linkages. Typically, a polynucleotide comprises at least three nucleosides. Usually oligonucleotides range in size from a few monomeric units, e.g. 3-4, to several hundreds of monomeric units. Whenever a polynucleotide such as an oligonucleotide is represented by a sequence of letters, such as “ATGCCTG,” it will be understood that the nucleotides are in 5′->3′ order from left to right and that “A” denotes deoxyadenosine, “C” denotes deoxycytidine, “G” denotes deoxyguanosine, and “T” denotes thymidine, unless otherwise noted. The letters A, C, G, and T may be used to refer to the bases themselves, to nucleosides, or to nucleotides comprising the bases, as is standard in the art.

The term “allele” as used herein refers to a genetic variation associated with a gene or a segment of DNA, i.e., one of two or more alternate forms of a DNA sequence occupying the same locus.

The term “locus” as used herein refers to a specific position on a chromosome or a nucleic acid molecule. Alleles of a locus are located at identical sites on homologous chromosomes.

The phrase “genomic variants” or “genome variants” denote a single or a grouping of sequences (in DNA or RNA) that have undergone changes as referenced against a particular species or sub-populations within a particular species due to mutations, recombination/crossover or genetic drift. Examples of types of genomic variants include, but are not limited to: single nucleotide polymorphisms (SNPs), copy number variations (CNVs), insertions/deletions (Indels), inversions, etc.

In various embodiments, genomic variants can be detected using a nucleic acid sequencing system and/or analysis of sequencing data. The sequencing workflow can begin with the test sample being sheared or digested into hundreds, thousands or millions of smaller fragments which are sequenced on a nucleic acid sequencer to provide hundreds, thousands or millions of sequence reads, such as nucleic acid sequence reads. Each read can then be mapped to a reference or target genome, and in the case of mate-pair fragments, the reads can be paired thereby allowing interrogation of repetitive regions of the genome. The results of mapping and pairing can be used as input for various standalone or integrated genome variant (for example, SNP, CNV, Indel, inversion, etc.) analysis tools.

As defined herein, “sample” and its derivatives, is used in its broadest sense and includes any specimen, culture and the like that is suspected of including a target. In some embodiments, the sample comprises DNA, RNA, PNA, LNA, chimeric, hybrid, or multiplex-forms of nucleic acids. The sample can include any biological, clinical, surgical, agricultural, atmospheric or aquatic-based specimen containing one or more nucleic acids. The term also includes any isolated nucleic acid sample such a genomic DNA, fresh-frozen or formalin-fixed paraffin-embedded nucleic acid specimen.

The phrase “sample genome” can denote a whole or partial genome of an organism.

As used herein, the terms “adapter” or “adapter and its complements” and their derivatives, refers to any linear oligonucleotide which can be ligated to a nucleic acid molecule of the disclosure. Optionally, the adapter includes a nucleic acid sequence that is not substantially complementary to the 3′ end or 5′ end of at least one target sequences within the sample. In some embodiments, the adapter is substantially non-complementary to the 3′ end or 5′ end of any target sequence present in the sample. In some embodiments, the adapter includes any single stranded or double-stranded linear oligonucleotide that is not substantially complementary to an amplified target sequence. In some embodiments, the adapter is substantially non-complementary to at least one, some or all of the nucleic acid molecules of the sample. In some embodiments, suitable adapter lengths are in the range of about 10-100 nucleotides, about 12-60 nucleotides and about 15-50 nucleotides in length. An adapter can include any combination of nucleotides and/or nucleic acids. In some aspects, the adapter can include one or more cleavable groups at one or more locations. In another aspect, the adapter can include a sequence that is substantially identical, or substantially complementary, to at least a portion of a primer, for example a universal primer. In some embodiments, the adapter can include a barcode or tag to assist with downstream cataloguing, identification or sequencing. In some embodiments, a single-stranded adapter can act as a substrate for amplification when ligated to an amplified target sequence, particularly in the presence of a polymerase and dNTPs under suitable temperature and pH.

As used herein, “DNA barcode” or “DNA tagging sequence” and its derivatives, refers to a unique short (e.g., 6-14 nucleotide) nucleic acid sequence within an adapter that can act as a ‘key’ to distinguish or separate a plurality of amplified target sequences in a sample. For the purposes of this disclosure, a DNA barcode or DNA tagging sequence can be incorporated into the nucleotide sequence of an adapter.

In some embodiments, the disclosure provides for amplification of multiple target-specific sequences from a population of target nucleic acid molecules. In some embodiments, the method comprises hybridizing one or more target-specific primer pairs to the target sequence, extending a first primer of the primer pair, denaturing the extended first primer product from the population of nucleic acid molecules, hybridizing to the extended first primer product the second primer of the primer pair, extending the second primer to form a double stranded product, and digesting the target-specific primer pair away from the double stranded product to generate a plurality of amplified target sequences. In some embodiments, the digesting includes partial digesting of one or more of the target-specific primers from the amplified target sequence. In some embodiments, the amplified target sequences can be ligated to one or more adapters. In some embodiments, adapters can include one or more DNA barcodes or tagging sequences. In some embodiments, amplified target sequences once ligated to an adapter can undergo a nick translation reaction and/or further amplification to generate a library of adapter-ligated amplified target sequences.

In some embodiments, the methods of the disclosure include selectively amplifying target sequences in a sample containing a plurality of nucleic acid molecules and ligating the amplified target sequences to at least one adapter and/or barcode. Adapters and barcodes for use in molecular biology library preparation techniques are well known to those of skill in the art. The definitions of adapters and barcodes as used herein are consistent with the terms used in the art. For example, the use of barcodes allows for the detection and analysis of multiple samples, sources, tissues or populations of nucleic acid molecules per multiplex reaction. A barcoded and amplified target sequence contains a unique nucleic acid sequence, typically a short 6-15 nucleotide sequence, that identifies and distinguishes one amplified nucleic acid molecule from another amplified nucleic acid molecule, even when both nucleic acid molecules minus the barcode contain the same nucleic acid sequence. The use of adapters allows for the amplification of each amplified nucleic acid molecule in a uniformed manner and helps reduce strand bias. Adapters can include universal adapters or propriety adapters both of which can be used downstream to perform one or more distinct functions. For example, amplified target sequences prepared by the methods disclosed herein can be ligated to an adapter that may be used downstream as a platform for clonal amplification. The adapter can function as a template strand for subsequent amplification using a second set of primers and therefore allows universal amplification of the adapter-ligated amplified target sequence. In some embodiments, selective amplification of target nucleic acids to generate a pool of amplicons can further comprise ligating one or more barcodes and/or adapters to an amplified target sequence. The ability to incorporate barcodes enhances sample throughput and allows for analysis of multiple samples or sources of material concurrently.

As used herein, a “targeted panel” refers to a set of target-specific primers that are designed for selective amplification of target gene sequences in a sample. In some embodiments, following selective amplification of at least one target sequence, the workflow further includes nucleic acid sequencing of the amplified target sequence.

As used herein, “target sequence” or “target gene sequence” and its derivatives, refers to any single or double-stranded nucleic acid sequence that can be amplified or synthesized according to the disclosure, including any nucleic acid sequence suspected or expected to be present in a sample. In some embodiments, the target sequence is present in double-stranded form and includes at least a portion of the particular nucleotide sequence to be amplified or synthesized, or its complement, prior to the addition of target-specific primers or appended adapters. Target sequences can include the nucleic acids to which primers useful in the amplification or synthesis reaction can hybridize prior to extension by a polymerase. In some embodiments, the term refers to a nucleic acid sequence whose sequence identity, ordering or location of nucleotides is determined by one or more of the methods of the disclosure.

As used herein, “target-specific primer” and its derivatives, refers to a single stranded or double-stranded polynucleotide, typically an oligonucleotide, that includes at least one sequence that is at least 50% complementary, typically at least 75% complementary or at least 85% complementary, more typically at least 90% complementary, more typically at least 95% complementary, more typically at least 98% or at least 99% complementary, or identical, to at least a portion of a nucleic acid molecule that includes a target sequence. In such instances, the target-specific primer and target sequence are described as “corresponding” to each other. In some embodiments, the target-specific primer is capable of hybridizing to at least a portion of its corresponding target sequence (or to a complement of the target sequence); such hybridization can optionally be performed under standard hybridization conditions or under stringent hybridization conditions. In some embodiments, the target-specific primer is not capable of hybridizing to the target sequence, or to its complement, but is capable of hybridizing to a portion of a nucleic acid strand including the target sequence, or to its complement. In some embodiments, a forward target-specific primer and a reverse target-specific primer define a target-specific primer pair that can be used to amplify the target sequence via template-dependent primer extension. Typically, each primer of a target-specific primer pair includes at least one sequence that is substantially complementary to at least a portion of a nucleic acid molecule including a corresponding target sequence but that is less than 50% complementary to at least one other target sequence in the sample. In some embodiments, amplification can be performed using multiple target-specific primer pairs in a single amplification reaction, wherein each primer pair includes a forward target-specific primer and a reverse target-specific primer, each including at least one sequence that substantially complementary or substantially identical to a corresponding target sequence in the sample, and each primer pair having a different corresponding target sequence.

The phrase “next generation sequencing,” or NGS, refers to sequencing technologies having increased throughput as compared to traditional Sanger- and capillary electrophoresis-based approaches, for example with the ability to generate hundreds of thousands of relatively small sequence reads at a time. Some examples of next generation sequencing techniques include, but are not limited to, sequencing by synthesis, sequencing by ligation, and sequencing by hybridization. Ultra-high throughput nucleic acid sequencing systems incorporating NGS technologies typically produce a large number of short sequence reads. Sequence processing methods should desirably assemble and/or map a large number of reads quickly and efficiently, such as to minimize use of computational resources. For example, data arising from sequencing of a mammalian genome can result in tens or hundreds of millions of reads that typically need to be assembled before they can be further analyzed to determine their biological, diagnostic and/or therapeutic relevance.

As used herein, the term “fully map” means that the sequence reads for an amplicon maps from end to end to a given reference sequence. The term “partially map” means that only a portion of the sequence read for the amplicon maps to a given reference sequence.

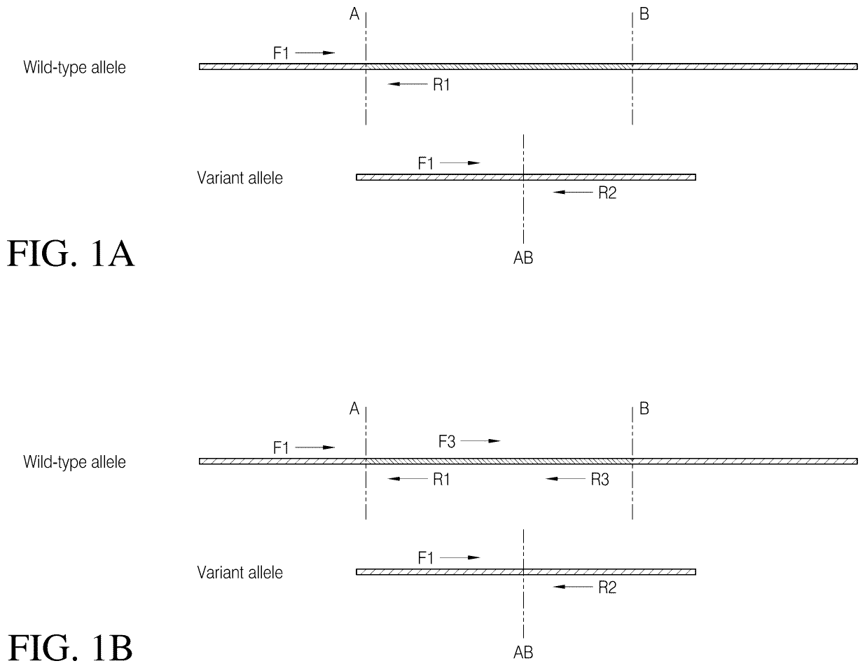

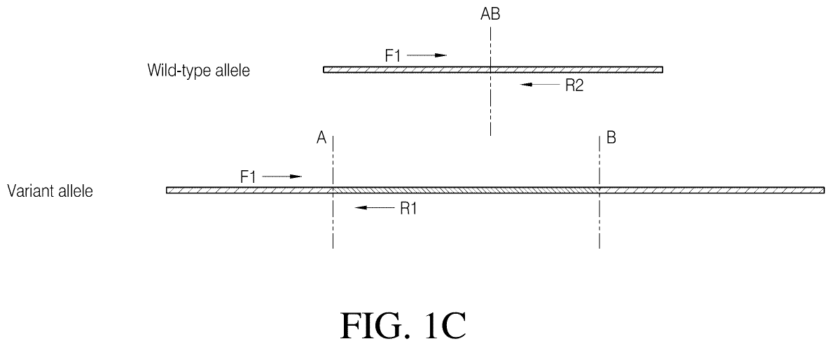

FIGS. 1A-1C give examples of possible forward and reverse primer positions in wild-type alleles and variant alleles for types of structural variants. FIG. 1A gives an example of a wild-type allele and variant allele for a deletion structural variant. The deletion region is between breakpoints A and B of the reference genome. The forward primer F1 is in a position on 5′ side of breakpoint A. The reverse primer R1 is in a position on 3′ side of breakpoint A. When amplifying a nucleic acid sample for an individual having the deletion structural variant using primers F1 and R2, the reads would fully map to the variant allele target region, or contig, in the modified reference sequence spanning both sides of point AB. When amplifying a nucleic acid sample for an individual having the wild-type allele using primers F1 and R1, the reads would fully map to a wild-type allele target region in the modified reference sequence spanning both sides of breakpoint A. FIG. 1B gives an example where there are more than two amplicons for a deletion structural variant. In this example, a forward primer F3 and reverse primer R3 are positioned within the deletion region between breakpoints A and B. When amplifying a nucleic acid sample for an individual having the wild-type allele using primers F3 and R3, the reads would fully map to the wild-type allele target region in the modified reference sequence, i.e., a target region between breakpoints A and B. FIG. 1C gives an example of a wild-type allele and a variant allele for an insertion structural variant. The insertion region is between points A and B of the variant allele. In this example, the forward primer F1 and the reverse primer R2 are in positions on each side of the breakpoint AB in the wild-type allele. For the variant allele, the forward primer F1 is in a position on 5′ side of the point A and reverse primer R1 is in position in the insertion region on the on 3′ side of the point A. When amplifying a nucleic acid sample for an individual having the wild-type allele using primers F1 and R2, the amplicons would fully map to the wild-type allele target region in the modified reference sequence spanning both sides of breakpoint AB. When amplifying a nucleic acid sample for an individual having the insertion structural variant allele using primers F1 and R1, the amplicons would fully map to the variant allele target region in the modified reference sequence spanning both sides of breakpoint A. The primers are designed in such a manner that sufficient numbers of sequence reads span the breakpoints of interest.

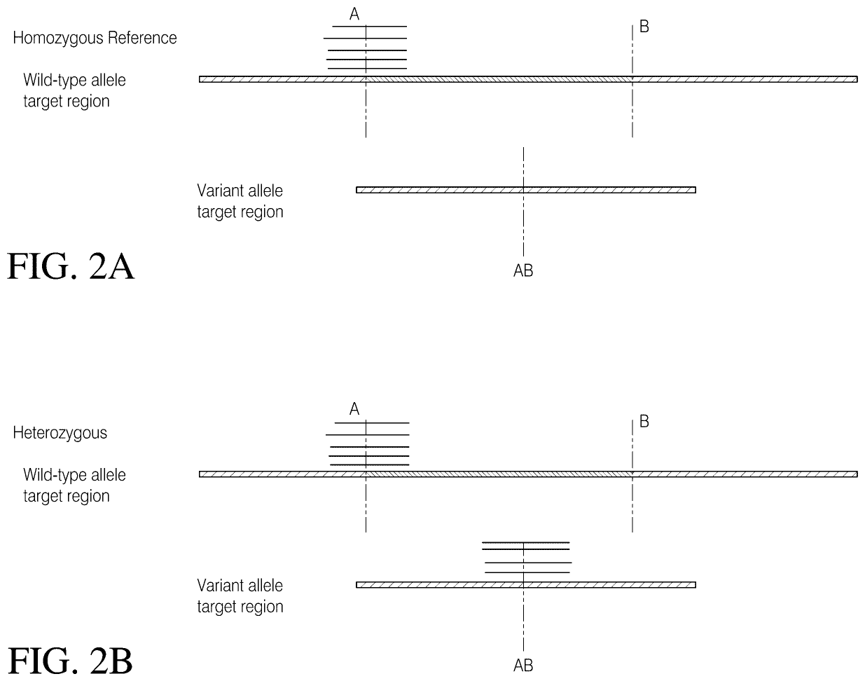

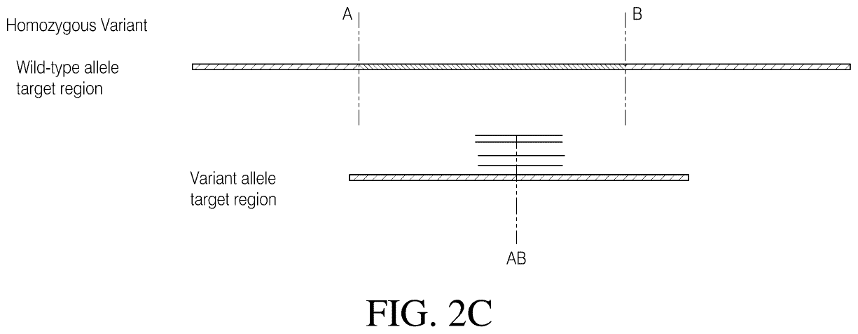

FIGS. 2A-2C give examples of alignments of sequence reads with the wild-type allele target region and the variant allele target region for various genotypes of a deletion structural variant. FIG. 2A shows an example of a homozygous wild-type genotype, where all of the amplicons are fully mapped to the wild-type allele target region in the modified reference sequence and span the breakpoint. FIG. 2B shows an example of a heterozygous genotype, where some reads are fully mapped to the wild-type allele target region and other reads are fully mapped to the variant allele target region in the modified reference sequence. FIG. 2C shows an example of a homozygous deletion structural variant genotype, where all of the reads are fully mapped to the variant allele target region in the modified reference sequence.

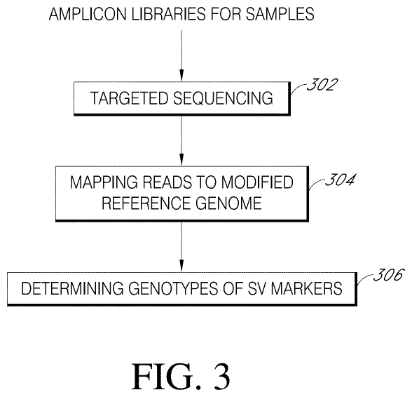

FIG. 3 is a block diagram of an example process for determining genotypes of structural variant markers. Selectively amplifying nucleic acid sequences at targeted locations in the sample genome by a panel targeting structural variant marker regions may produce amplicon libraries for one or more test samples. For example, the Applied Biosystems AgriSeq HTS Library Kit (Thermo Fisher Scientific catalog nos. A34144 and A34143) provides high-throughput preparation of amplicon libraries for targeted genotyping-by-sequencing (GBS) applications in agrigenomics. The amplicon libraries of multiple samples may be barcoded to distinguish different samples that are sequenced simultaneously in a single sequencing run. In step 302, the amplicons of the sample libraries are sequenced by a nucleic acid sequencing device, such as a next generation sequencing device, to produce a plurality of sequence reads. A plurality of samples from one or more organisms may be sequenced simultaneously in a single run to produce a plurality of sequence reads. In step 304, the sequence reads are mapped to a modified reference genome for the organism to produce the aligned sequence reads. The modified reference genome, or modified reference sequence, may include a wild-type target region (which is already present in the original reference genome) and a structural variant target region, or contig. The structural variant contig may be generated by applying the particular type of structural variation to the reference genome. For example, a deletion variant contig can be generated by deleting the portion of the reference sequence located between the breakpoints. For example, an insertion variant contig can be generated by inserting the insertion sequence into the reference sequence between the breakpoints. For example, an inversion contig can be generated by inverting the portion of the reference sequence located between the breakpoints. The aligned sequence reads for the modified reference genome, including both the original reference genome and structural variant contigs may be stored in a file using a BAM file format, for example. FIG. 2A shows an example where all of the aligned sequence reads are fully mapped to the wild-type allele contig for the homozygous reference genotype. FIG. 2B shows an example where some of the sequence reads are fully mapped to the wild-type contig and other reads are fully mapped to the deletion structural variant contig for the heterozygous genotype. FIG. 2C shows an example where all of the sequence reads are fully mapped to the deletion structural variant contig for the homozygous deletion structural variant genotype. The aligned sequence reads may correspond to a plurality of targeted structural variant marker regions. The SV genotyping step 306 may provide the genotypes for the structural variants using the aligned sequence reads.

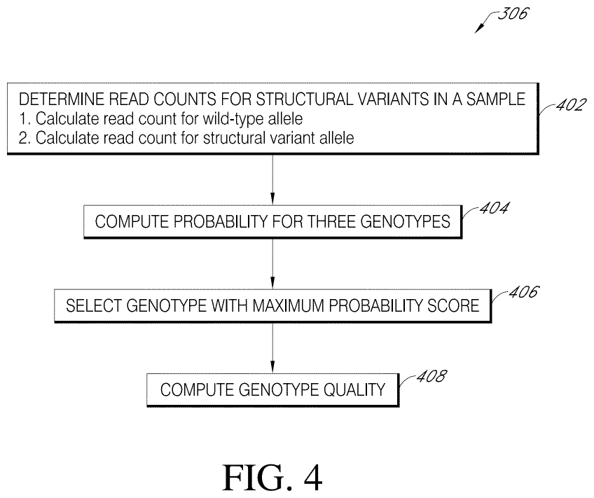

FIG. 4 is a block diagram of an example process for determining the genotype for each structural variant marker in each sample. In step 402, the read counts supporting the wild-type allele and SV allele are determined for each SV marker in each sample. A probability model fit to the read count data observed at a position in a given sequence read may be generated for hypothesized alleles. An a posteriori probability distribution f_{APP} is given by the expression:

$${f}_{APP} ( {p}_{H} = f ) = Beta ( k + 1 , n - k + 1 ) \qquad ( 1 )$$

where (p_{H}=f) is the probability that an allele frequency equals f for the hypothesized allele, n is the total read depth for wild-type and variant alleles combined for a given SV marker, k is the number of reads supporting the alternate allele, and (n−k) is the number of reads supporting a wild-type allele. In some cases there may be multiple amplicons for an allele, as shown by the primer pairs F1/R1 and F3/R3 in FIG. 1B. For the situation of multiple amplicons for the wild-type allele, the read depth for the wild-type allele is the normalized read depths of the multiple amplicons and n is the normalized read depth for the wild-type allele plus read depth for the variant allele. For example, if there are two amplicons for the wild-type allele having read depths x1 and x2, respectively, then the normalized read depth is (x1+x2)/2 and n=k+ (x1+x2)/2. The function Beta represents the probability model fit to the observed read count data for the hypothesized alleles. For example, the probability model may be a beta-binomial distribution.

In step 404, the probability for each possible genotype hypothesis is calculated. For example, these probabilities may be calculated as follows.

For the probability, pop, of the homozygous wild-type genotype:

$${p}_{0 / 0} = {\int }_{f = 0}^{0.1} {f}_{APP} ( {p}_{H} = f ) \qquad ( 2 )$$

For the probability, p_{0/1}, of the heterozygous genotype:

$${p}_{0 / 1} = {\int }_{f = 0.1}^{0.9} {f}_{APP} ( {p}_{H} = f ) \qquad ( 3 )$$

For the probability, p_{1/1}, of the homozygous structural variant genotype:

$${p}_{1 / 1} = {\int }_{f = 0.9}^{1} {f}_{APP} ( {p}_{H} = f ) \qquad ( 4 )$$

The limits of integration in equations (2) through (4) are default values. Allele frequency boundary parameters may be used to set the limits of integration. The boundary parameter is the allele frequency value of a boundary in the a posteriori probability distribution f_{APP} (p_{H}=f) for genotyping a structural variant associated with a specific SV marker.

The allele frequency boundary parameters, and the related limits of integration in equations (2) through (4), may be set to other values. For example, the limits of integration may be customized for each SV marker. A user may set the values of the allele frequency boundary parameters.

In step 406, the estimated genotype associated with the highest probability value, or probability score, is selected. The estimated genotype i is the one which has the highest probability value.

$$i = {Max}_{i = \{ 0 / 0 , 0 / 1 , 1 / 1 \}} \{ {p}_{i} \} \qquad ( 5 )$$

The estimated genotype may indicate the estimated genotype associated with the SV marker and sample.

In step 408, the genotype quality may be calculated. The estimated genotype quality is the probability of estimating an incorrect genotype. The probability of an incorrect genotype may be calculated as −10 log₁₀(summation of probabilities supporting other genotypes). For example, if the estimated genotype is 0/0, then the genotype quality is given by:

$$Genotype quality = - 10 {log}_{10} ( {p}_{0 / 1} + {p}_{1 / 1} ) \qquad ( 6 )$$

A threshold may be applied to the genotype quality to determine whether a genotype call should be made. A minimum variant score parameter may provide a threshold value for genotype quality. The genotype quality is determined by equation (6). If the genotype score is lower than that the minimum variant score, then the genotype call will be a “NO CALL”. The minimum variant score may be set to a value greater than 0. For example, the minimum variant score may be set to a default value of 10. The minimum variant score parameter may be set by the user.

A minimum threshold may be applied to the coverage for the location of the SV marker to determine whether a genotype call for the SV should be made. A minimum coverage parameter may provide a minimum threshold value for the coverage. The minimum coverage threshold may be applied to the sum of the read coverage for wild-type allele and the read coverage for the alternate/SV allele. The minimum coverage parameter is greater than zero. If the coverage is lower than minimum coverage parameter value, then a “NO CALL” is assigned. The minimum coverage parameter may be set to an integer value greater than 0 by the user. For example, the minimum coverage parameter may be set to a default value of 20. The minimum coverage parameter may be set to a user-defined value for a particular marker. There may be different minimum coverage parameters for different SV markers.

A maximum threshold may be applied to the SV marker. A maximum coverage parameter may provide a threshold, or a ceiling, for the maximum coverage for each of the SV amplicons designed for the marker which will be used in the genotyping model. For example, the maximum coverage parameter may be set to a default value of 400. For example, if the SV amplicon has a coverage of 1000 reads, then the genotyping model may assume that the coverage was 400 reads. The maximum coverage parameter may be set to an integer value greater than 0 by the user. The maximum coverage parameter may be set to a user-defined value for a particular SV marker. There may be different minimum coverage parameters for different SV markers. The maximum coverage parameter may control an extreme imbalance in read counts for wild-type and variant alleles caused by differences in amplification efficiency of designed primer pairs for wild-type allele and variant allele.

A minimum structural variant frequency parameter indicates the minimum frequency of the structural variant to be reported. For example, the minimum SV frequency parameter has a default value of 0.10. The minimum SV frequency parameter may be a decimal number between 0 and 1. The minimum SV frequency parameter may be used to set the allele frequency boundary parameters and the related limits of integration in equations (2), (3) and (4). The values of the allele frequency boundary parameters may be set to 0, minimum SV frequency parameter, 1−minimum SV frequency parameter, and 1.0. For example, the minimum SV frequency parameter value of 0.1 was used to set the allele frequency boundary parameters shown in equations (2), (3) and (4), which are 0, 0.1, 0.9, and 1.0. The minimum structural variant frequency parameter may be set by the user. Values of the minimum structural variant frequency parameter are adjustable on a per structural variant marker basis. Increasing values of the minimum SV frequency parameter may make SV calling become less sensitive but more specific.

A minimum bp extend parameter may allow consideration of the reads which extend beyond minimum bp extend parameter on 5′ and 3′ sides of the SV breakpoint. The minimum bp extend parameter may be an integer value greater than 0. For example, a default value for the minimum bp extend parameter may be set to 5. For the default value of 5, reads that extend at least 5 bases beyond the breakpoint in 5′ direction and at least 5 bases beyond the breakpoint in 3′ direction are included for determining the read count in step 402. Increasing values of the minimum bp extend parameter make SV calling become less sensitive but more specific.

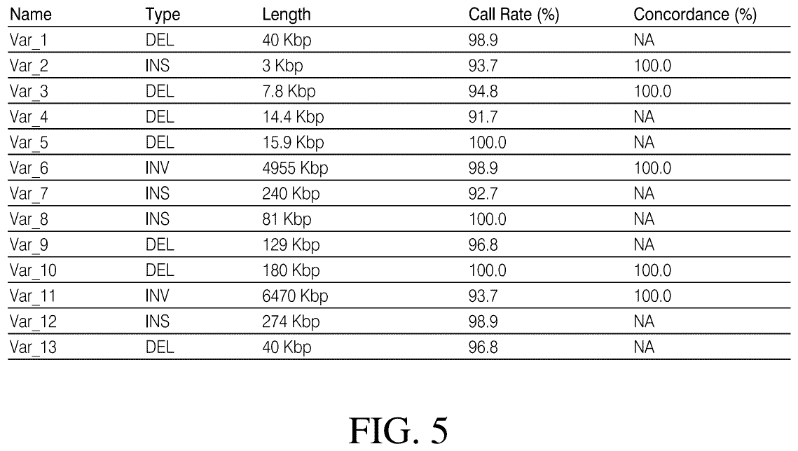

FIG. 5 gives a table of results of tests for genotyping canine SV markers. In these tests, 13 structural variant markers were tested in 96 samples simultaneously in a single sequencing run. Both insertion structural variants (INS) and deletion structural variants (DEL) were tested. The lengths of the structural variants ranged from 81 bp to 4955 Kbp (kilobase pair). The call rate for the genotype and concordance of the genotype call with the truth data, where available, were determined. The results show 100% concordance compared to available truth data for five samples. The average call rate was 97%. These results show that primer design and downstream analysis were not impacted by the size of the structural variant.

The high call rates across multiple samples with varying structural variant sizes indicate the reproducibility and flexibility of the present method. The targeted GBS using the present method is effective for genotyping diverse marker types simultaneously using the same workflow.

The embodiments disclosed herein may achieve improved accuracy of genotyping calls for structural variants over a wide range of lengths and types of structural variants relative to conventional approaches. As shown in the table of test results in FIG. 5, the accuracies of genotyping calls, or call rates, for structural variants concordant with known truth were consistent for structural variant lengths from 180 bp to 4955 Kbp. The conventional approaches suffer from a number of technical problems and limitations, including inconsistent accuracies for different lengths and types of structural variants. The present embodiments enable parameter adjustment at a marker level to improve the genotyping accuracy for each marker in a sample and provide consistent accuracy across various lengths of structural variants. The embodiments disclosed herein can determine structural variant genotype on a per sample per marker basis and are effective for insertion, deletion and inversion structural variants across a wide range of structural variant lengths.

Various ones of the embodiments disclosed herein may improve upon conventional approaches to achieve the technical advantages of consistent accuracies for detecting the genotypes of structural variants for different lengths and types of structural variants. Technical advantages of the embodiments described herein include improved accuracy in the estimate of genotypes for insertion, deletion and inversion structural variants. Such technical advantages are not achievable by routine and conventional approaches, and users of systems and methods including such embodiments may benefit from these advantages. The technical features of the embodiments disclosed herein are thus unconventional in the field of deriving genotypes of a structural variants, including insertion, deletion and inversion structural variants across a wide range of structural variant lengths.

Accordingly, the embodiments of the present disclosure serve a technical purpose, such as deriving genotype estimates for structural variants, defined as having at least 50 bp in length, on a per sample per marker basis. In particular, the present disclosure provides technical solutions to technical problems, including but not limited to providing consistent accuracies of genotype estimates for structural variants including insertion, deletion and inversion structural variants across a wide range of structural variant lengths.

In various embodiments, nucleic acid sequence data can be generated using various techniques, platforms or technologies, including, but not limited to: capillary electrophoresis, microarrays, ligation-based systems, polymerase-based systems, hybridization-based systems, direct or indirect nucleotide identification systems, pyrosequencing, ion- or pH-based detection systems, electronic signature-based systems, fluorescent-based detection systems, single molecule methods, etc.

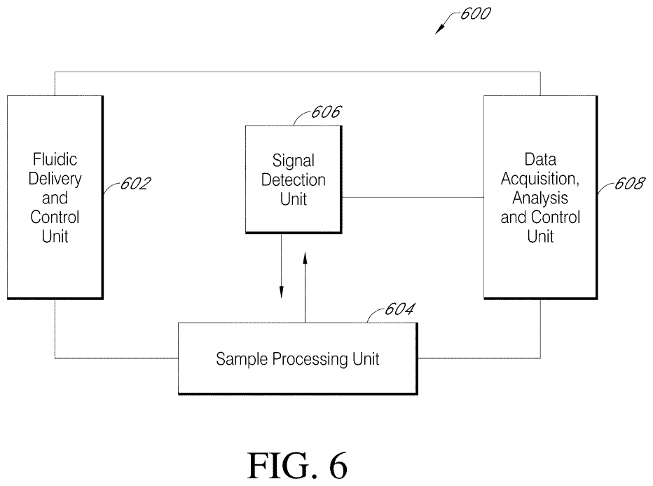

Various embodiments of nucleic acid sequencing platforms, such as a nucleic acid sequencer, can include components as displayed in the block diagram of FIG. 6. According to various embodiments, sequencing instrument 600 can include a fluidic delivery and control unit 602, a sample processing unit 604, a signal detection unit 606, and a data acquisition, analysis and control unit 608. Various embodiments of instrumentation, reagents, libraries and methods used for next generation sequencing are described in U.S. Patent Application Publication No. 2009/0127589 and No. 2009/0026082. Various embodiments of instrument 600 can provide for automated sequencing that can be used to gather sequence information from a plurality of sequences in parallel, such as substantially simultaneously.

In various embodiments, the fluidics delivery and control unit 602 can include reagent delivery system. The reagent delivery system can include a reagent reservoir for the storage of various reagents. The reagents can include RNA-based primers, forward/reverse DNA primers, oligonucleotide mixtures for ligation sequencing, nucleotide mixtures for sequencing-by-synthesis, optional ECC oligonucleotide mixtures, buffers, wash reagents, blocking reagent, stripping reagents, and the like. Additionally, the reagent delivery system can include a pipetting system or a continuous flow system which connects the sample processing unit with the reagent reservoir.

In various embodiments, the sample processing unit 604 can include a sample chamber, such as flow cell, a substrate, a micro-array, a multi-well tray, or the like. The sample processing unit 604 can include multiple lanes, multiple channels, multiple wells, or other means of processing multiple sample sets substantially simultaneously. Additionally, the sample processing unit can include multiple sample chambers to enable processing of multiple runs simultaneously. In particular embodiments, the system can perform signal detection on one sample chamber while substantially simultaneously processing another sample chamber. Additionally, the sample processing unit can include an automation system for moving or manipulating the sample chamber.

In various embodiments, the signal detection unit 606 can include an imaging or detection sensor. For example, the imaging or detection sensor can include a CCD, a CMOS, an ion sensor, such as an ion sensitive layer overlying a CMOS, a current detector, or the like. The signal detection unit 606 can include an excitation system to cause a probe, such as a fluorescent dye, to emit a signal. The expectation system can include an illumination source, such as arc lamp, a laser, a light emitting diode (LED), or the like. In particular embodiments, the signal detection unit 606 can include optics for the transmission of light from an illumination source to the sample or from the sample to the imaging or detection sensor. Alternatively, the signal detection unit 606 may not include an illumination source, such as for example, when a signal is produced spontaneously as a result of a sequencing reaction. For example, a signal can be produced by the interaction of a released moiety, such as a released ion interacting with an ion sensitive layer, or a pyrophosphate reacting with an enzyme or other catalyst to produce a chemiluminescent signal. In another example, changes in an electrical current can be detected as a nucleic acid passes through a nanopore without the need for an illumination source.

In various embodiments, data acquisition analysis and control unit 608 can monitor various system parameters. The system parameters can include temperature of various portions of instrument 600, such as sample processing unit or reagent reservoirs, volumes of various reagents, the status of various system subcomponents, such as a manipulator, a stepper motor, a pump, or the like, or any combination thereof.

It will be appreciated by one skilled in the art that various embodiments of instrument 600 can be used to practice variety of sequencing methods including ligation-based methods, sequencing by synthesis, single molecule methods, nanopore sequencing, and other sequencing techniques.

In various embodiments, the sequencing instrument 600 can determine the sequence of a nucleic acid, such as a polynucleotide or an oligonucleotide. The nucleic acid can include DNA or RNA, and can be single stranded, such as ssDNA and RNA, or double stranded, such as dsDNA or a RNA/cDNA pair. In various embodiments, the nucleic acid can include or be derived from a fragment library, a mate pair library, a ChIP fragment, or the like. In particular embodiments, the sequencing instrument 600 can obtain the sequence information from a single nucleic acid molecule or from a group of substantially identical nucleic acid molecules.

In various embodiments, sequencing instrument 600 can output nucleic acid sequencing read data in a variety of different output data file types/formats, including, but not limited to: *.fasta, *.csfasta, *seq.txt, *qseq.txt, *.fastq, *.sff, *prb.txt, *.sms, *srs and/or *.qv.

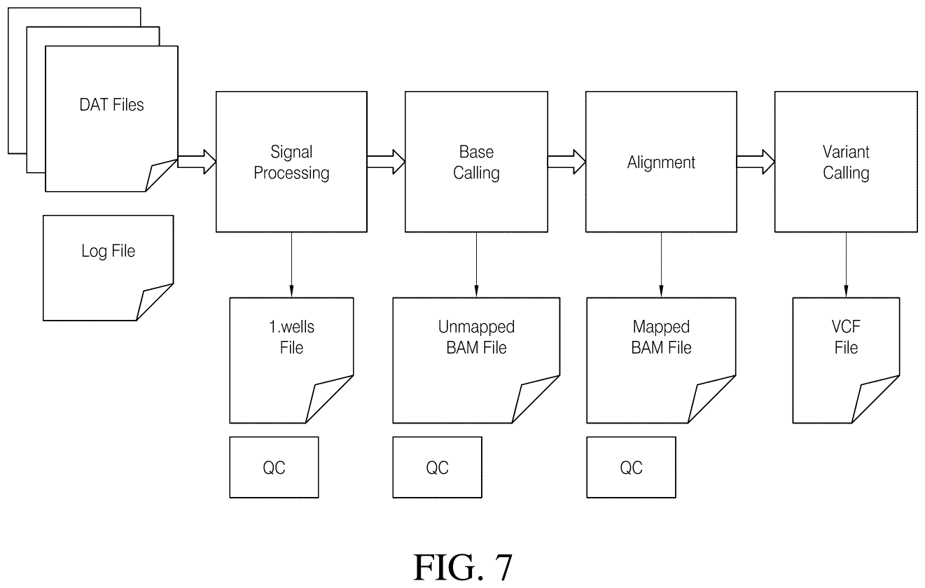

FIG. 7 is a block diagram of an analysis pipeline for signal data obtained from a nucleic acid sequencing instrument. The sequencing instrument generates raw data files (DAT, or .dat, files) during a sequencing run for an assay. Signal processing may be applied to raw data to generate incorporation signal measurement data for files, such as the 1.wells files, which are transferred to the server FTP location along with the log information of the run. The signal processing step may derive background signals corresponding to wells. The background signals may be subtracted from the measured signals for the corresponding wells. The remaining signals may be fit by an incorporation signal model to estimate the incorporation at each nucleotide flow for each well. The output from the above signal processing is a signal measurement per well and per flow, that may be stored in a file, such as a 1.wells file.

In some embodiments, the base calling step may perform phase estimations, normalization, and runs a solver algorithm to identify best partial sequence fit and make base calls. The base sequences for the sequence reads are stored in unmapped BAM files. The base calling step may generate total number of reads, total number of bases, and average read length as quality control (QC) measures to indicate the base call quality. The base calls may be made by analyzing any suitable signal characteristics (e.g., signal amplitude or intensity). The signal processing and base calling for use with the present teachings may include one or more features described in U.S. Pat. Appl. Publ. No. 2013/0090860 published Apr. 11, 2013, U.S. Pat. Appl. Publ. No. 2014/0051584 published Feb. 20, 2014, and U.S. Pat. Appl. Publ. No. 2012/0109598 published May 3, 2012, each incorporated by reference herein in its entirety.

Once the base sequence for the sequence read is determined, the sequence reads may be provided to the alignment step, for example, in an unmapped BAM file. The alignment step maps the sequence reads to a reference genome to determine aligned sequence reads and associated mapping quality parameters. The alignment step may generate a percent of mappable reads as QC measure to indicate alignment quality. The alignment results may be stored in a mapped BAM file. Methods for aligning sequence reads for use with the present teachings may include one or more features described in U.S. Pat. Appl. Publ. No. 2012/0197623, published Aug. 2, 2012, incorporated by reference herein in its entirety.

The BAM file format structure is described in “Sequence Alignment/Map Format Specification,” Sep. 12, 2014 (github.com/samtools/hts-specs). As described herein, a “BAM file” refers to a file compatible with the BAM format. As described herein, an “unmapped” BAM file refers to a BAM file that does not contain aligned sequence read information and mapping quality parameters and a “mapped” BAM file refers to a BAM file that contains aligned sequence read information and mapping quality parameters.

The variant calling step may include detecting single-nucleotide polymorphisms (SNPs), insertions and deletions (InDels), multi-nucleotide polymorphisms (MNPs), and complex block substitution events. In various embodiments, a variant caller can be configured to communicate variants called for a sample genome as a *.vcf, *.gff, or *.hdf data file. The called variant information can be communicated using any file format as long as the called variant information can be parsed and/or extracted for analysis. The variant detection methods for use with the present teachings may include one or more features described in U.S. Pat. Appl. Publ. No. 2013/0345066, published Dec. 26, 2013, U.S. Pat. Appl. Publ. No. 2014/0296080, published Oct. 2, 2014, and U.S. Pat. Appl. Publ. No. 2014/0052381, published Feb. 20, 2014, each of which is incorporated by reference herein in its entirety.

According to various exemplary embodiments, one or more features of any one or more of the above-discussed teachings and/or exemplary embodiments may be performed or implemented using appropriately configured and/or programmed hardware and/or software elements. Determining whether an embodiment is implemented using hardware and/or software elements may be based on any number of factors, such as desired computational rate, power levels, heat tolerances, processing cycle budget, input data rates, output data rates, memory resources, data bus speeds, etc., and other design or performance constraints.

Examples of hardware elements may include processors, microprocessors, input(s) and/or output(s) (I/O) device(s) (or peripherals) that are communicatively coupled via a local interface circuit, circuit elements (e.g., transistors, resistors, capacitors, inductors, and so forth), integrated circuits, application specific integrated circuits (ASIC), programmable logic devices (PLD), digital signal processors (DSP), field programmable gate array (FPGA), logic gates, registers, semiconductor device, chips, microchips, chip sets, and so forth. The local interface may include, for example, one or more buses or other wired or wireless connections, controllers, buffers (caches), drivers, repeaters and receivers, etc., to allow appropriate communications between hardware components. A processor is a hardware device for executing software, particularly software stored in memory. The processor can be any custom made or commercially available processor, a central processing unit (CPU), an auxiliary processor among several processors associated with the computer, a semiconductor based microprocessor (e.g., in the form of a microchip or chip set), a macroprocessor, or generally any device for executing software instructions. A processor can also represent a distributed processing architecture. The I/O devices can include input devices, for example, a keyboard, a mouse, a scanner, a microphone, a touch screen, an interface for various medical devices and/or laboratory instruments, a bar code reader, a stylus, a laser reader, a radio-frequency device reader, etc. Furthermore, the I/O devices also can include output devices, for example, a printer, a bar code printer, a display, etc. Finally, the I/O devices further can include devices that communicate as both inputs and outputs, for example, a modulator/demodulator (modem; for accessing another device, system, or network), a radio frequency (RF) or other transceiver, a telephonic interface, a bridge, a router, etc.

Examples of software may include software components, programs, applications, computer programs, application programs, system programs, machine programs, operating system software, middleware, firmware, software modules, routines, subroutines, functions, methods, procedures, software interfaces, application program interfaces (API), instruction sets, computing code, computer code, code segments, computer code segments, words, values, symbols, or any combination thereof. A software in memory may include one or more separate programs, which may include ordered listings of executable instructions for implementing logical functions. The software in memory may include a system for identifying data streams in accordance with the present teachings and any suitable custom made or commercially available operating system (O/S), which may control the execution of other computer programs such as the system, and provides scheduling, input-output control, file and data management, memory management, communication control, etc.

According to various exemplary embodiments, one or more features of any one or more of the above-discussed teachings and/or exemplary embodiments may be performed or implemented using appropriately configured and/or programmed non-transitory machine-readable medium or article that may store an instruction or a set of instructions that, if executed by a machine, may cause the machine to perform a method and/or operations in accordance with the exemplary embodiments. Such a machine may include, for example, any suitable processing platform, computing platform, computing device, processing device, computing system, processing system, computer, processor, scientific or laboratory instrument, etc., and may be implemented using any suitable combination of hardware and/or software. The machine-readable medium or article may include, for example, any suitable type of memory unit, memory device, memory article, memory medium, storage device, storage article, storage medium and/or storage unit, for example, memory, removable or non-removable media, erasable or non-erasable media, writeable or re-writeable media, digital or analog media, hard disk, floppy disk, read-only memory compact disc (CD-ROM), recordable compact disc (CD-R), rewriteable compact disc (CD-RW), optical disk, magnetic media, magneto-optical media, removable memory cards or disks, various types of Digital Versatile Disc (DVD), a tape, a cassette, etc., including any medium suitable for use in a computer. Memory can include any one or a combination of volatile memory elements (e.g., random access memory (RAM, such as DRAM, SRAM, SDRAM, etc.)) and nonvolatile memory elements (e.g., ROM, EPROM, EEROM, Flash memory, hard drive, tape, CDROM, etc.). Moreover, memory can incorporate electronic, magnetic, optical, and/or other types of storage media. Memory can have a distributed architecture where various components are situated remote from one another, but are still accessed by the processor. The instructions may include any suitable type of code, such as source code, compiled code, interpreted code, executable code, static code, dynamic code, encrypted code, etc., implemented using any suitable high-level, low-level, object-oriented, visual, compiled and/or interpreted programming language.

According to various exemplary embodiments, one or more features of any one or more of the above-discussed teachings and/or exemplary embodiments may be performed or implemented at least partly using a distributed, clustered, remote, or cloud computing resource.

According to various exemplary embodiments, one or more features of any one or more of the above-discussed teachings and/or exemplary embodiments may be performed or implemented using a source program, executable program (object code), script, or any other entity comprising a set of instructions to be performed. When a source program, the program can be translated via a compiler, assembler, interpreter, etc., which may or may not be included within the memory, so as to operate properly in connection with the O/S. The instructions may be written using (a) an object oriented programming language, which has classes of data and methods, or (b) a procedural programming language, which has routines, subroutines, and/or functions, which may include, for example, C, C++, R, Python, Pascal, Basic, Fortran, Cobol, Perl, Java, and Ada.

According to various exemplary embodiments, one or more of the above-discussed exemplary embodiments may include transmitting, displaying, storing, printing or outputting to a user interface device, a computer readable storage medium, a local computer system or a remote computer system, information related to any information, signal, data, and/or intermediate or final results that may have been generated, accessed, or used by such exemplary embodiments. Such transmitted, displayed, stored, printed or outputted information can take the form of searchable and/or filterable lists of runs and reports, pictures, tables, charts, graphs, spreadsheets, correlations, sequences, and combinations thereof, for example.

### EXAMPLES

Example 1 is a method for determining genotypes of structural variants in a sample genome, including: amplifying nucleic acid sequences at targeted locations in the sample genome by a panel targeting a plurality of structural variant markers of the sample to generate a plurality of sequence reads; mapping the plurality of sequence reads to a modified reference genome to produce a plurality of aligned sequence reads, wherein the modified reference genome includes a wild-type target region and a structural variant target region; for each structural variant marker: determining a read count for a wild-type allele and a read count for a structural variant allele; determining a probability for each possible genotype, wherein possible genotypes include a homozygous wild-type genotype, a heterozygous genotype and a homozygous structural variant genotype; and selecting the genotype associated with a maximum probability value to provide an estimated genotype corresponding to the structural variant marker of the sample.

Example 2 includes the subject matter of Example 1, and further includes determining a genotype quality based on a summation of probabilities supporting other possible genotypes.

Example 3 includes the subject matter of Example 2, and further specifies that the determining the genotype quality further comprises calculating a log₁₀ of the summation of the probabilities and multiplying the log₁₀ of the summation of probabilities by (−10).

Example 4 includes the subject matter of Example 2, and further includes applying a threshold to the genotype quality to determine whether a genotype call will be made, wherein if the genotype quality is less than the threshold then the genotype call will be a “NO CALL”.

Example 5 includes the subject matter of Example 1, and further includes applying a threshold to a sum of the read count for the wild-type allele and the read count for the structural variant allele to determine whether a genotype call will be made for the structural variant marker, wherein if the sum is less than the threshold then the genotype call will be a “NO CALL”.

Example 6 includes the subject matter of Example 5, and further specifies that the threshold is adjustable on a per structural variant marker basis.

Example 7 includes the subject matter of Example 1, and further specifies that determining the probability for each possible genotype is based on an a posteriori probability distribution of allele frequencies for a hypothesized allele.

Example 8 includes the subject matter of Example 7, and further specifies that determining the probability for each possible genotype further comprises integrating the a posteriori probability distribution of allele frequencies between limits of integration, wherein variant allele frequency boundary parameters set the limits of integration for each possible genotype corresponding to the structural variant marker.

Example 9 includes the subject matter of Example 1, and further includes applying minimum structural variant frequency parameter that indicates a minimum variant allele frequency for a structural variant to be called.

Example 10 includes the subject matter of Example 9, and further specifies that the minimum structural variant frequency parameter has a value of 0.1.

Example 11 includes the subject matter of Example 9, and further specifies that values of the minimum structural variant frequency parameter are adjustable on a per structural variant marker basis.

Example 12 includes the subject matter of Example 1, and further specifies that the step of determining a read count for a wild-type allele and a read count for a structural variant allele further comprises comparing a maximum coverage parameter to the read count for the wild-type allele and the read count for the structural variant allele, wherein a read count greater than the maximum coverage parameter is set to a default value.

Example 13 includes the subject matter of Example 1, and further specifies that the step of determining a read count for a wild-type allele and a read count for a structural variant allele further comprises excluding sequence reads that do not extend on both 5′ and 3′ sides of a breakpoint by at least a minimum number of bases.

Example 14 includes the subject matter of Example 1, and further specifies that the structural variant region of the modified reference genome includes a deletion variant contig.

Example 15 includes the subject matter of Example 1, and further specifies that the structural variant region of the modified reference genome includes an insertion variant contig.

Example 16 includes the subject matter of Example 1, and further specifies that the structural variant region of the modified reference genome includes an inversion contig.

Example 17 is a system for determining genotypes of structural variants in a sample genome, including: a machine-readable memory; and a processor configured to execute machine-readable instructions, which are configured to, when executed by the processor, cause the system to perform steps, comprising: receiving, at the processor, a plurality of sequence reads produced by amplifying nucleic acid sequences at targeted locations in the sample genome by a panel targeting a plurality of structural variant markers of the sample to generate a plurality of sequence reads; mapping the plurality of sequence reads to a modified reference genome to produce a plurality of aligned sequence reads, wherein the modified reference genome includes a wild-type target region and a structural variant target region; for each structural variant marker: determining a read count for a wild-type allele and a read count for a structural variant allele; determining a probability for each possible genotype, wherein possible genotypes include a homozygous wild-type genotype, a heterozygous genotype and a homozygous structural variant genotype; and selecting the genotype associated with a maximum probability value to provide an estimated genotype corresponding to the structural variant marker of the sample.

Example 18 includes the subject matter of Example 17, wherein the steps further include determining a genotype quality based on a summation of probabilities supporting other possible genotypes.

Example 19 includes the subject matter of Example 18, and further specifies that determining the genotype quality further comprises calculating a log₁₀ of the summation of the probabilities and multiplying the log₁₀ of the summation of probabilities by (−10).

Example 20 includes the subject matter of Example 18, and further includes applying a threshold to the genotype quality to determine whether a genotype call will be made, wherein if the genotype quality is less than the threshold then the genotype call will be a “NO CALL”.

Example 21 includes the subject matter of Example 17, and further specifies that the steps further include applying a threshold to a sum of the read count for the wild-type allele and the read count for the structural variant allele to determine whether a genotype call will be made for the structural variant marker, wherein if the sum is less than the threshold then the genotype call will be a “NO CALL”.

Example 22 includes the subject matter of Example 21, and further specifies that the threshold is adjustable on a per structural variant marker basis.

Example 23 includes the subject matter of Example 17, and further specifies that determining the probability for each possible genotype is based on an a posteriori probability distribution of allele frequencies for a hypothesized allele

Example 24 includes the subject matter of Example 23, and further specifies that determining the probability for each possible genotype further comprises integrating the a posteriori probability distribution of allele frequencies between limits of integration, wherein variant allele frequency boundary parameters set the limits of integration for each possible genotype corresponding to the structural variant marker.

Example 25 includes the subject matter of Example 17, and further specifies that the steps further include applying minimum structural variant frequency parameter that indicates a minimum variant allele frequency for a structural variant to be called.

Example 26 includes the subject matter of Example 25, and further specifies that the minimum structural variant frequency parameter has a value of 0.1.

Example 27 includes the subject matter of Example 25, and further specifies that values of the minimum structural variant frequency parameter are adjustable on a per structural variant marker basis.

Example 28 includes the subject matter of Example 17, and further specifies that the step of determining a read count for a wild-type allele and a read count for a structural variant allele further comprises comparing a maximum coverage parameter to the read count for the wild-type allele and the read count for the structural variant allele, wherein a read count greater than the maximum coverage parameter is set to a default value.

Example 29 includes the subject matter of Example 17, and further specifies that the step of determining a read count for a wild-type allele and a read count for a structural variant allele further comprises excluding sequence reads that do not extend on both 5′ and 3′ sides of a breakpoint by at least a minimum number of bases.

Example 30 includes the subject matter of Example 17, and further specifies that the structural variant region of the modified reference genome includes a deletion variant contig.

Example 31 includes the subject matter of Example 17, and further specifies that the structural variant region of the modified reference genome includes an insertion variant contig.

Example 32 includes the subject matter of Example 17, and further specifies that the structural variant region of the modified reference genome includes an inversion contig.

Example 33 is a non-transitory machine-readable storage medium comprising instructions which are configured to, when executed by a processor, cause the processor to perform a method for determining genotypes of structural variants in a sample genome, including: receiving, at the processor, a plurality of sequence reads produced by amplifying nucleic acid sequences at targeted locations in the sample genome by a panel targeting a plurality of structural variant markers of the sample to generate a plurality of sequence reads; mapping the plurality of sequence reads to a modified reference genome to produce a plurality of aligned sequence reads, wherein the modified reference genome includes a wild-type target region and a structural variant target region; for each structural variant marker: determining a read count for a wild-type allele and a read count for a structural variant allele; determining a probability for each possible genotype, wherein possible genotypes include a homozygous wild-type genotype, a heterozygous genotype and a homozygous structural variant genotype; and selecting the genotype associated with a maximum probability value to provide an estimated genotype corresponding to the structural variant marker of the sample.

Example 34 includes the subject matter of Example 33, further including instructions which cause the processor to perform the method that further includes determining a genotype quality based on a summation of probabilities supporting other possible genotypes.

Example 35 includes the subject matter of Example 34, and further specifies that the determining the genotype quality further comprises calculating a log₁₀ of the summation of the probabilities and multiplying the log₁₀ of the summation of probabilities by (−10).

Example 36 includes the subject matter of Example 34, further including instructions which cause the processor to perform the method that further includes applying a threshold to the genotype quality to determine whether a genotype call will be made, wherein if the genotype quality is less than the threshold then the genotype call will be a “NO CALL”.

Example 37 includes the subject matter of Example 33, further including instructions which cause the processor to perform the method that further includes applying a threshold to a sum of the read count for the wild-type allele and the read count for the structural variant allele to determine whether a genotype call will be made for the structural variant marker, wherein if the sum is less than the threshold then the genotype call will be a “NO CALL”.

Example 38 includes the subject matter of Example 37, and further specifies that the threshold is adjustable on a per structural variant marker basis.

Example 39 includes the subject matter of Example 33, and further specifies that determining the probability for each possible genotype is based on an a posteriori probability distribution of allele frequencies for a hypothesized allele.

Example 40 includes the subject matter of Example 39, and further specifies that determining the probability for each possible genotype further comprises integrating the a posteriori probability distribution of allele frequencies between limits of integration, wherein variant allele frequency boundary parameters set the limits of integration for each possible genotype corresponding to the structural variant marker.

Example 41 includes the subject matter of Example 33, further including instructions which cause the processor to perform the method, that further includes applying minimum structural variant frequency parameter that indicates a minimum variant allele frequency for a structural variant to be called.

Example 42 includes the subject matter of Example 41, and further specifies that the minimum structural variant frequency parameter has a value of 0.1.

Example 43 includes the subject matter of Example 41, and further specifies that values of the minimum structural variant frequency parameter are adjustable on a per structural variant marker basis.

Example 44 includes the subject matter of Example 33, and further specifies that the step of determining a read count for a wild-type allele and a read count for a structural variant allele further comprises comparing a maximum coverage parameter to the read count for the wild-type allele and the read count for the structural variant allele, wherein a read count greater than the maximum coverage parameter is set to a default value.

Example 45 includes the subject matter of Example 33, and further specifies that the step of determining a read count for a wild-type allele and a read count for a structural variant allele further comprises excluding sequence reads that do not extend on both 5′ and 3′ sides of a breakpoint by at least a minimum number of bases.

Example 46 includes the subject matter of Example 33, and further specifies that the structural variant region of the modified reference genome includes a deletion variant contig.

Example 47 includes the subject matter of Example 33, and further specifies that the structural variant region of the modified reference genome includes an insertion variant contig.

Example 48 includes the subject matter of Example 33, and further specifies that the structural variant region of the modified reference genome includes an inversion contig.

What is claimed is:

1. A method for determining genotypes of structural variants in a sample genome, comprising:
amplifying nucleic acid sequences at targeted locations in the sample genome by a panel targeting a plurality of structural variant markers of the sample to generate a plurality of sequence reads;
mapping the plurality of sequence reads to a modified reference genome to produce a plurality of aligned sequence reads, wherein the modified reference genome includes a wild-type target region and a structural variant target region;
for each structural variant marker,
determining a read count for a wild-type allele and a read count for a structural variant allele;
determining a probability for each possible genotype, wherein possible genotypes include a homozygous wild-type genotype, a heterozygous genotype and a homozygous structural variant genotype; and
selecting the genotype associated with a maximum probability value to provide an estimated genotype corresponding to the structural variant marker of the sample.

2. The method of claim 1, further comprising determining a genotype quality based on a summation of probabilities supporting other possible genotypes.

3. The method of claim 2, wherein the determining the genotype quality further comprises calculating a log₁₀ of the summation of the probabilities and multiplying the log₁₀ of the summation of probabilities by (−10).

4. The method of claim 1, wherein the determining the probability for each possible genotype is based on an a posteriori probability distribution of allele frequencies for a hypothesized allele.

5. The method of claim 4, wherein the determining the probability for each possible genotype further comprises integrating the a posteriori probability distribution of allele frequencies between limits of integration, wherein variant allele frequency boundary parameters set the limits of integration for each possible genotype corresponding to the structural variant marker.

6. The method of claim 1, further comprising applying minimum structural variant frequency parameter that indicates a minimum variant allele frequency for a structural variant to be called.

7. The method of claim 6, wherein the minimum structural variant frequency parameter has a value of 0.1.

8. The method of claim 6, wherein values of the minimum structural variant frequency parameter are adjustable on a per structural variant marker basis.

9. The method of claim 1, wherein the step of determining a read count for a wild-type allele and a read count for a structural variant allele further comprises comparing a maximum coverage parameter to the read count for the wild-type allele and the read count for the structural variant allele, wherein a read count greater than the maximum coverage parameter is set to a default value.

10. The method of claim 1, wherein the step of determining a read count for a wild-type allele and a read count for a structural variant allele further comprises excluding sequence reads that do not extend on both 5′ and 3′ sides of a breakpoint by at least a minimum number of bases.

11. A system for determining genotypes of structural variants in a sample genome, comprising:
a machine-readable memory; and
a processor configured to execute machine-readable instructions, which are configured to, when executed by the processor, cause the system to perform steps, comprising:
receiving, at the processor, a plurality of sequence reads produced by amplifying nucleic acid sequences at targeted locations in the sample genome by a panel targeting a plurality of structural variant markers of the sample to generate a plurality of sequence reads;
mapping the plurality of sequence reads to a modified reference genome to produce a plurality of aligned sequence reads, wherein the modified reference genome includes a wild-type target region and a structural variant target region;
for each structural variant marker,
determining a read count for a wild-type allele and a read count for a structural variant allele;
determining a probability for each possible genotype, wherein possible genotypes include a homozygous wild-type genotype, a heterozygous genotype and a homozygous structural variant genotype; and
selecting the genotype associated with a maximum probability value to provide an estimated genotype corresponding to the structural variant marker of the sample.

12. The system of claim 11, wherein the steps further include determining a genotype quality based on a summation of probabilities supporting other possible genotypes.

13. The system of claim 12, wherein the determining the genotype quality further comprises calculating a log₁₀ of the summation of the probabilities and multiplying the log₁₀ of the summation of probabilities by (−10).

14. The system of claim 11, wherein the determining the probability for each possible genotype is based on an a posteriori probability distribution of allele frequencies for a hypothesized allele.

15. The system of claim 14, wherein the determining the probability for each possible genotype further comprises integrating the a posteriori probability distribution of allele frequencies between limits of integration, wherein variant allele frequency boundary parameters set the limits of integration for each possible genotype corresponding to the structural variant marker.

16. The system of claim 11, wherein the steps further include applying minimum structural variant frequency parameter that indicates a minimum variant allele frequency for a structural variant to be called.

17. The system of claim 16, wherein the minimum structural variant frequency parameter has a value of 0.1.

18. The system of claim 16, wherein values of the minimum structural variant frequency parameter are adjustable on a per structural variant marker basis.

19. The system of claim 11, wherein the step of determining a read count for a wild-type allele and a read count for a structural variant allele further comprises comparing a maximum coverage parameter to the read count for the wild-type allele and the read count for the structural variant allele, wherein a read count greater than the maximum coverage parameter is set to a default value.

20. The system of claim 11, wherein the step of determining a read count for a wild-type allele and a read count for a structural variant allele further comprises excluding sequence reads that do not extend on both 5′ and 3′ sides of a breakpoint by at least a minimum number of bases.
